(19) United States
(12) Patent Application Publication
(10) Pub. No.: US 2011/0138521 A1
(43) Pub. Date: Jun. 16, 2011

(54) Party goggles

(76) Inventor: Bruce Riggs, Helendale, CA (US)

(21) Appl. No.: 12/927,974

(22) Filed: Nov. 30, 2010

Related U.S. Application Data
(60) Provisional application No. 61/283,185, filed on Nov. 30, 2009.

(51) Int. Cl. A61F 9/02

(52) U.S. Cl. 2/452

(57) ABSTRACT

The present invention provides novelty eye goggles comprising an adjustable elastic rubber strap attached to the clear plastic frames of standard, vented safety goggles, with two hollowed-out 12 ounce aluminum beer cans affixed to the eye-sockets, protruding outward from the frames, suspended horizontally and running parallel to the ground. The cans themselves feature a number of brand logos and advertisements,

### CLAIM OF PRIORITY

This patent application claims priority under 35 USC 119 (e) (1) from U.S. Provisional Patent Application Ser. No. 61/283,185 filed Nov. 11, 2009, of common inventorship herewith entitled, “Party Goggles.”

### FIELD OF THE INVENTION

The present invention pertains to the field of novelty items, and more specifically to the field of eye goggles.

### BACKGROUND OF THE INVENTION

The prior art has put forth several designs for eye goggles. Among these are:

U.S. Pat. No. 6,178,561 to Chen-Sun Cheng describes safety goggles having a lens frame force-fitted in a primary frame.

U.S. Pat. No. 6,309,270 to Samuel K. Harwell, IV and Craig C. Williamson describes swim gear including a headpiece which covers the forward portion of the wearer's head and supports a pair of spaced lenses therein.

U.S. Pat. No. 7,062,797 to Basimah Khulusi describes a multi-purpose goggle for protecting the eyes of the wearer in industrial and sporting environments and against glare when the wearer is viewing a computer screen for extended periods of time, for housing a virtual reality display and for use by pilots engaged in training for their instrument flight rating.

None of these prior art references describe the present invention.

### SUMMARY OF THE INVENTION

It is an object of the present invention to provide novelty party eye goggles.

### BRIEF DESCRIPTION OF THE DRAWINGS

FIG. 1 is a front angled perspective view of the device of the present invention.

FIG. 2 is a front perspective view of the device of the present invention.

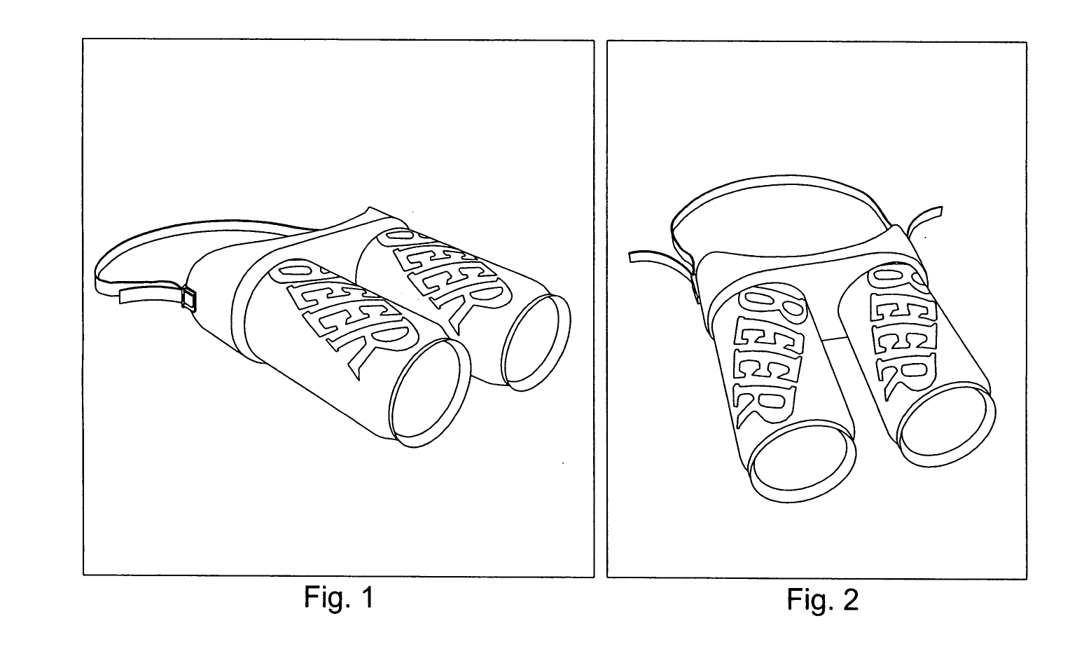

### DETAILED DESCRIPTION OF THE INVENTION

The present invention provides a novel party item hereinafter referred to as Beer Goggles or Party goggles.

Simply stated, the Party Goggles are the literal personification of the mythical Beer Goggles “worn” by so many intoxicated adults on so many beer-fueled weekend nights all across the nation, and for that matter, the world. Manufactured by the Miscellaneous Manufacturing industries, Standard Industrial Code 3999, the Party Goggles look exactly like one imagines. They feature an adjustable elastic rubber strap attached to the clear plastic frames of standard, vented safety goggles, think science fair, not swim meet, with two hollowed-out 12 ounce aluminum beer cans affixed to the eye-sockets, protruding outward from the frames, suspended horizontally and running parallel to the ground. The cans themselves feature a number of brand logos and advertisements, from the ubiquitous, classic party staples like Bud Light and Heineken, to more exotic overseas imports like Guinness Stout or Kirin Ichiban.

The top and bottom portions of the cans are cut out and rolled or capped for smoothness, then glued or fastened via a plastic or rubber gasket, leaving only the hollow cylindrical portions to look through, much like binoculars, minus the actual functionality, of course, but far more amusing.

Use of the Party Goggles is extremely simple and straightforward. First, the user throws a good, old-fashioned booze-soaked shindig. If that isn't a possibility, you can just as easily crash someone else's. Next, spend some time enjoying the party; have a few drinks, get to know everybody. Then, at the opportune moment, ideally sometime after the first keg has been tapped, but before anyone has yet had to resort to drinking the decade-old Battles & James hidden in the back of the fridge since New Years' Eve '88, the wearer retrieves the Goggles from his conveyance, places them on his or her head, and then proceeds to strut or stumble, depending on the level of consumption, around the party like everything was normal and let the laughs ensue.

The Party Goggles present a number of distinct benefits and advantages. Foremost, simply putting on the Party Goggles allows the user to resurrect an otherwise lifeless party from the bowels of mediocrity, or gives the wearer the power to kick an average party into high-gear. How many times have we all been stuck at a fetid fete, marooned at the beer-stained end of a crowded couch, uncomfortably sipping a flat beer and hoping against hope that someone would do or say something interesting, something memorable. That is precisely what the Party Goggles provide, a fun, memorable evening among friends. It takes a certain kind of person, a rare individual indeed, to be able to cold-open a conversation with other random guests, to have the innate ability to jump-start a sagging saturnalia, to bring both old friends and perfect strangers together in frivolous celebration or, in short, to be the “life of the party.” With the Party Goggles, everyone enjoys that same elusive ability to unite a group of people in shared laughter and collective merriment. After the user first puts on the Party Goggles, it soon becomes apparent that such an innovative and clever sight-gag has the potential to pull some heavy-duty laughs, and the capacity to spread like a California wildfire. Before too long, every person at the party is talking about the “hilarious Beer Goggle-Guy,” wondering where you are so they can ask you where in the world you get those things, so they can go out and buy a pair for themselves. What some people fail to realize is that everybody at a party wants to be the center of attention, even the quiet ones. Some just lack the courage, or perhaps the gimmick, that allows them to do so. That is precisely what the Party Goggles provide. They allow the boisterous jock or shameless socialite to maintain their clamorous reputation, while letting even the most reserved couch-dweller have their own hilarious moment in the spotlight.

A cleverly-conceived new novelty item made to let “party people” freely express their sense of individuality and help crank up the festivities, the Party Goggles proudly display, in a very literal way, the figurative eyewear we all have put on at one point or another. A gut-busting sight-gag aimed at those who might find themselves in a raucous roadhouse, hectic house party or fun family get-together, the Party Goggles should find a wide and receptive market among both the swarming barflies and regular, fun-loving folks.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. Novelty eye goggles, comprising: an adjustable elastic rubber strap attached to clear plastic frames of standard, vented safety goggles, comprising two hollowed-out 12 ounce aluminum beer cans affixed to the eye-sockets, protruding outward from the frames, suspended horizontally and running parallel to the ground.

2. The novelty eye goggles of claim 1 wherein the cans are imprinted with brand logos or advertisements.
